(19) United States
(12) Patent Application Publication
(10) Pub. No.: US 2008/0319277 A1
(43) Pub. Date: Dec. 25, 2008

(54) SLEEP DISORDER MONITORING AND DIAGNOSTIC SYSTEM

(75) Inventor: Donald Carmon Bradley, Kanata (CA)

Correspondence Address: BORDEN LADNER GERVAIS LLP;Anne Kinsman, WORLD EXCHANGE PLAZA, 100 QUEEN STREET SUITE 1100, OTTAWA, ON, K1P 1J9, CA

(73) Assignee: BRAEBON MEDICAL CORPORATION, Carp, ON (CA)

(21) Appl. No.: 11/572,518

(22) PCT Filed: Jun. 12, 2006
(86) PCT No.: PCT/CA06/00955

§ 371 Date: Jan. 23, 2007

Related U.S. Application Data
(60) Provisional application No. 60/689,535, filed on Jun. 13, 2005.

(51) Int. Cl. A61B 5/00

(52) U.S. Cl. 600/301

(57) ABSTRACT

A portable or wearable system for monitoring and diagnosing sleep disorders, such as sleep apnea, and an associated method of monitoring and diagnosis. The device which can be used for the detection, assessment, diagnosis and pre-diagnosis (screening) of sleep apnea, as well as other sleep-related disorders associated with sleep apnea, such as hypopnea, snoring and abnormal cardiac rhythms. The device preferably samples, stores and records sound at a frequency of 1000 Hz and higher to allow for an accurate analysis of the subject's condition to be carried out. Memory is provided in the device to store at least six hours of continuous data. Data collected by the device can be downloaded to an external computing device for later use and analysis by a medical professional.

### FIELD OF THE INVENTION

The present invention relates generally to physiological monitoring and diagnosis devices. In particular, the present invention relates to a wearable physiological device for the monitoring and diagnosis of sleep disorders, such as sleep apnea.

### BACKGROUND OF THE INVENTION

As the detrimental physical effects of sleep-related disorders become more and more known, the need to accurately diagnose such disorders becomes more acute. Reduced productivity, reduced quality of life and even death have been shown to be directly attributed to sleep-related disorders. These sleep-related disorders include sleep apnea (where a subject stops breathing for ten or more seconds repeatedly through the night), upper airway resistance, snoring, and abnormal cardiac rhythms. Sleep apnea alone has been linked to a loss of billions of dollars on the GDP of the United States. Sleep disorders, and in particular sleep apnea, have also recently been shown to be a major influence on cardiac problems. As a result, cardiologists are now looking for ways to evaluate an individual as to their cardiac performance while they are asleep.

Proper diagnosis of sleep apnea is important because the preferred methods for treating most respiratory sleep disorders require interventionist measures to be carried out on the subject. These interventionist measures can consist of blowing air into a subject's nose or mouth so as to eliminate or reduce the closing of the breathing passage in the back of the throat (Continuous Positive Airway Pressure or CPAP), the use of an oral appliance that holds the lower jaw of a subject in a forward position thus eliminating or reducing the closing of the airway passage, and surgery to remove excess or re-shape the uvula. The two surgical procedures commonly used to treat sleep apnea are uvulopalatopharyngoplasty (UPPP) and palatopharyngoplasty (PPP). These procedures are attempts to create a permanent, non-collapsing oropharyngeal airway. There are several technical variations to these procedures but all make use of the same basic UPPP procedure. It should be noted that quite often additional or repeated UPPP or PPP surgery or tonsillectomy or septoplasty may be required until an acceptable reduction in the severity of the sleep-related disorder is achieved.

Respiratory sleep-related disorders usually occur due to a cerebral (central) problem, a restriction to the airflow (obstructive) or a combination of the two (mixed). The therapies described above only work on obstructive and mixed disorders. Diagnosing which type of disorder requires not only an analysis of the subject's respiratory airflow, but also an analysis of the subject's respiratory effort. Obstructive, central and mixed events are all characterized by a change in the volume of air moving in and out of the subject. Obstructive events can be characterized by a paradoxical movement of the chest and abdomen, thus demonstrating that the subject is attempting to breath, but that there is an obstruction. A further indication of restrictions in airflow can be obtained by monitoring snoring sounds.

Diagnosing sleep disorders requires studying a subject while they are asleep for an extended period of time, usually from four to ten hours. Devices known in the art for diagnosing sleep-related disorders typically require a subject to be connected by numerous wires to one or more diagnostic devices that sit either on the subject's nightstand or in another room. Current polysomnography systems for the diagnosis of sleep apnea, or other sleep-related disorders, typically require an expensive overnight sleep study that is administered and analyzed by a trained technician. The limited availability of sleep centers coupled with the high capital expense has resulted in a growing number of subjects awaiting proper diagnosis and treatment.

A conventional full overnight polysomnography includes recording of the following signals: electroencephalogram (EEG), submental electromyogram (EMG), electrooculogram (EOG), respiratory airflow (oronasal flow monitors), respiratory effort (plethysmography), oxygen saturation (oximetry), electrocardiography (ECG), electromyography (EMG), snoring sounds, and body position. These signals offer a relatively complete collection of parameters from which respiratory events may be identified and sleep apnea may be reliably diagnosed.

Proper diagnosis of a sleep disorder usually requires that sleep studies be performed for more than one night as it has been shown that there is a first night effect where the subject will not sleep properly due to the change in sleep environment. For proper diagnosis, a subject should have as normal a sleep as possible. Traveling to a clinic/hospital, and being hooked up to many sensors that are in turn connected to immovable equipment can all severely restrict a subject's ability to sleep as they normally would. By contrast, allowing a subject to sleep in their usual bed with a minimum of sensors and equipment attached, and no restriction to their movement can provide more accurate information on a subject, and may decrease the number of sleep sessions that must be monitored for proper diagnosis.

Attempts have been made in the past to provide wearable sleep disorder monitoring and diagnosis devices. However, such devices are limited to collection of a limited number of diagnostic signals (e.g. airflow only), and do not collect auditory signals for snoring, bruxism or breathing sounds at high enough sampling rates to allow for a proper analysis of the subject's condition to be carried out. In either case, insufficient data may be collected for full and proper diagnosis of a subject's sleep disorder. In addition, the sensors of previously proposed devices are often integrated with the monitoring and recording unit, and thus are not easily reconfigurable or exchangeable.

It is, therefore, desirable to provide a sleep disorder diagnostic or monitoring device that is wearable and measures a plurality of blood oxygen saturation (SpO2), pulse rate, internal body position, airflow, chest respiratory effort, abdomen respiratory effort and acoustic signals indicative of snoring or labored breathing.

### SUMMARY OF THE INVENTION

It is an object of the present invention to obviate or mitigate at least one disadvantage of previous sleep monitoring and sleep disorder diagnosis systems.

In a first aspect, the present invention provides a sleep disorder monitoring and diagnostic device. The device comprises a processing and recording unit to be worn by a subject for monitoring and diagnosis of a sleep disorder in the subject. The processing and recording unit has a plurality of connectors to permit reconfigurable attachment of various physiological sensors for sensing physiological conditions of the subject; processing means for sampling and processing signals from the physiological sensors; and storage means for recording the sampled and processed signals.

According to various embodiments of this aspect, the plurality of connectors include two or more different connector types, selected from, or example, leur lock connectors, auxiliary connectors, and pin keyed connectors. The physiological sensors can include a microphone, and operate at a sound sample rate is at least 1000 Hz. The physiological sensors can also include oxyhemoglobin sensors, pulse rate sensors, electrocardiogram (ECG) sensors, and respiratory effort sensors. The device can further include an airflow pressure sensor for use with a nasal cannula, and a body position detector.

In accordance with a further aspect, the present invention provides a sleep disorder and diagnostic device kit. The kit comprises a plurality of physiological sensors for sensing physiological conditions of a subject; and a processing and recording unit to be worn by a subject for monitoring and diagnosis of a sleep disorder in the subject, the processing and recording unit having a plurality of connectors to permit reconfigurable attachment of the plurality of physiological sensors, a processing means for sampling and processing signals from the physiological sensors, and a storage means for recording the sampled and processed signals. The kit can be a single use kit.

According to yet another aspect, the present invention provides a sleep disorder monitoring and diagnostic method. The method comprises steps of attaching a processing and recording unit to a subject, the processing and recording unit having a plurality of connectors to permit reconfigurable attachment of various physiological sensors; connecting a plurality of physiological sensors to the processing and recording unit, including a microphone to detect sound related to breathing and snoring; and sampling signals from the plurality of physiological sensors, including sampling sound, via the microphone, at at least 1000 Hz.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 shows an embodiment of a sleep disorder monitoring and diagnosis system according to the present invention;

FIG. 2 is an interior view of the processing and recording unit of FIG. 1;

FIG. 3 is a bottom view of the processing and recording unit of FIG. 1;

FIG. 4 is a block diagram of the processing and recording circuitry of the processing and recording unit of FIG. 1;

FIG. 5 shows a further embodiment of a sleep disorder monitoring and diagnosis system according to the present invention; and

FIG. 6 shows yet another embodiment of a sleep disorder monitoring and diagnosis system according to the present invention.

### DETAILED DESCRIPTION

Generally, the present invention provides a portable or wearable system for monitoring and diagnosing sleep disorders, such as sleep apnea, and an associated method of monitoring and diagnosis. The present invention is a wearable physiological diagnostic device which can be used for the detection, assessment, diagnosis and pre-diagnosis (screening) of sleep apnea, as well as other sleep-related disorders associated with sleep apnea, such as hypopnea, snoring and abnormal cardiac rhythms. The present invention preferably samples, stores and records sound at about a frequency of 1000 Hz and higher to allow for an accurate analysis of the subject's condition to be carried out. Preferably, sufficient memory is provided in the device to store at least six hours of continuous data. Data collected by the present invention can be downloaded to an external computing device for later use and analysis by a medical professional.

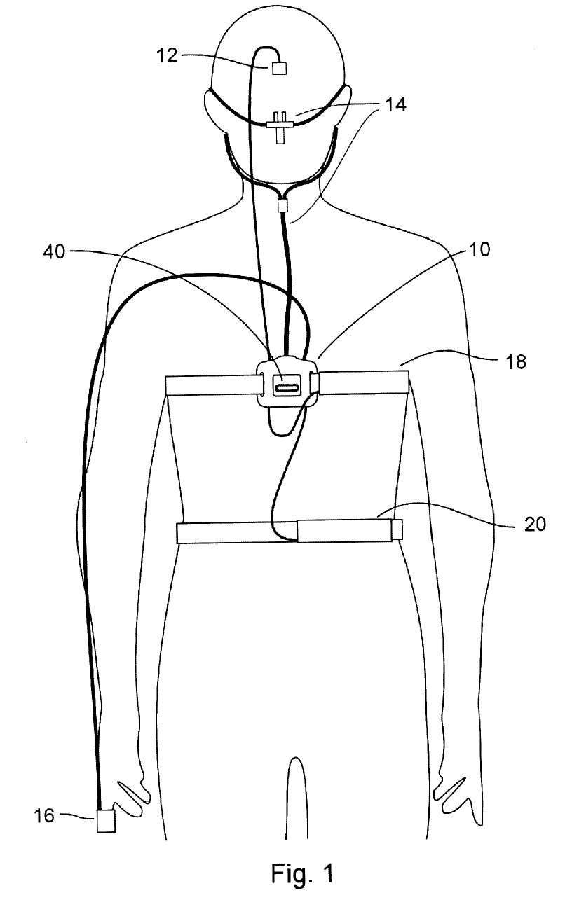

As shown in FIG. 1, the present invention is comprised of a portable or wearable processing and recording unit 10 that can be worn by a subject on the chest (as shown) or elsewhere on the subject. The processing and recording unit 10 can be connected to sensing devices, such as a microphone 12 for sampling snoring/breathing sounds, a nasal cannula 14 for sensing airflow, a SpO2/pulse finger sensor 16 for measuring pulse and blood oxygen saturation, and respiratory effort sensors 18 and 20 for measuring chest and abdominal respiratory effort, respectively. Processing and recording unit 10 can affixed to the subject, or attached to the subject via a strap that goes through the gull wings 22 and around the subject's thorax, or arm or other extremity.

The processing and recording unit 10 of the present invention is a self-contained battery-powered medical diagnostic sampling, amplifying, digitizing, storage, recording and communication device. In a preferred embodiment, a battery, such as a conventional alkaline battery, lithium hydride or nickel cadmium battery, is used as a power source.

The processing and recording unit 10 is capable of collecting audio sounds (i.e. snoring, bruxism and breathing sounds) at sampling rates of 1000 Hz or higher. In addition to sampling snoring/bruxism/breathing sounds, the processing and recording unit 10 of the present invention can be used to measure or monitor any one or more of the following: blood oxygen saturation, pulse rate, body position, activity, airflow, chest respiratory effort and abdomen respiratory effort. The processing and recording unit 10 is preferably mounted to a subject's thorax by belts strung through the gull wings 22 on the sides of the processing and recording unit 10.

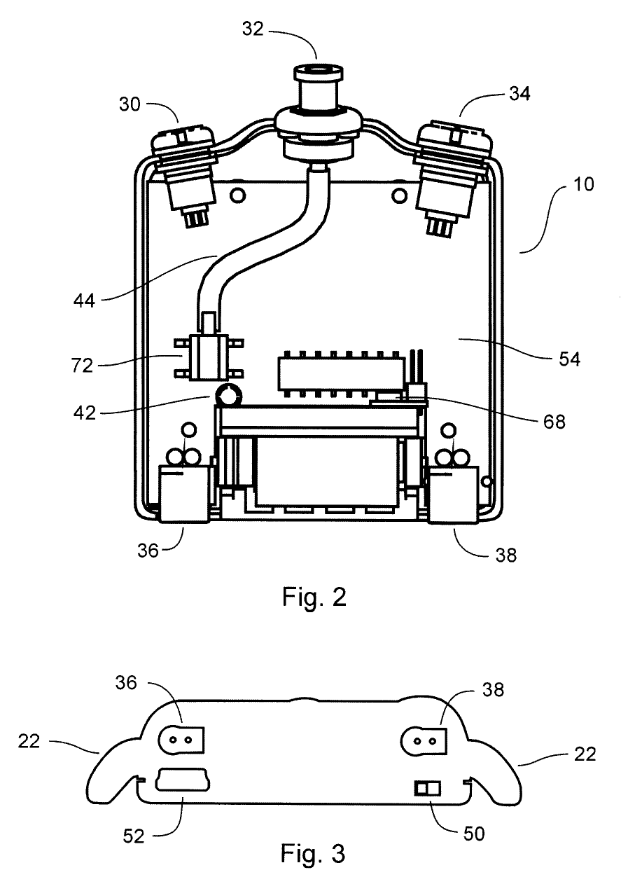

As shown in FIG. 2, an embodiment of the processing and recording unit 10 of the present invention includes a dual purpose auxiliary (AUX) connector 30, a leur lock connector 32 for connecting to a nasal or nasal/oral cannula, an SpO2 connector 34, a chest respiratory effort connector 36, an abdomen respiratory effort connector 38, a set/event button 40 (shown in FIG. 1), and a status LED 42. The particular connectors and their arrangement are exemplary only, and it is fully contemplated by the inventor that any connectors or other interfaces that permit communication with an auxiliary or remote sensor unit can be integrated into the device. Preferably, the connections can permit specific sensors to be attached in such a manner as to minimize subject discomfort and allow sound data to be collected reliably at sampling rates of 1000 Hz or higher.

The set button 40 can be depressed by the subject to provide a timestamp for an event such as lights off or lights on, which is then recorded and stored in a memory of the unit 10. The status LED 42 is used to indicate if the processing and recording unit 10 is operating properly or if there is a condition existing in the processing and recording unit 10, such as low battery power or sensor disconnection.

FIG. 3 shows a bottom view of the processing and recording unit 10 of the present invention. An optional ON/OFF switch 50 is provided, as well as a communication port 52. By using the ON/OFF switch 50, the subject can control when the processing and recording unit 10 is to commence sampling and storing physiological data when the ON/OFF switch 50 is in the ON position. The processing and recording unit 10 can be set up or initialized to start sampling and storing data at a certain date and time thus avoiding the requirement for an ON/OFF switch. The communication port 52, such as a serial or universal serial bus (USB) communication port, is used to interface the processing and recording unit 10 to an external computing device such as a printer, monitor, or external storage device, such as for the downloading of data recorded by the processing and recording unit 10. The communication port 52 may also be configured to accept an electronic key that informs the processing and recording unit 10 as to how many studies are to be performed. This electronic key can then be used to monitor the number of studies actually performed to ensure that the unit is not used more than permitted. The gull wing shape of the illustrated embodiment, provides the device with a functional advantage in that the device can be mounted to one of the effort sensors, such as chest respiratory effort connector 36, thus reducing the number of straps that the monitoring subject needs to attach. Although this is advantageous, it should not be considered to be restrictive, as devices of the present invention could be implemented without making use of this feature.

When an electronic key is included, the manufacturer can limit the number of uses of the device and ensure that the subject is receiving new single use devices each time the unit is used. The electronic key will prevent the clinician from reusing single use devices, and as such is another aspect for subject safety. The electronic key can, for example, consist of a microprocessor that is configured with a number that indicates the number of uses it is programmed for. When the electronic key is inserted into communication port 52, the processing and recording unit 10 detects the electronic key and turns on the status LED 42 to a solid green while it is reading the number of uses programmed into the key. The processing and recording unit 10 then erases the number on the electronic key and flashes green until the electronic key is removed. The processing and recording unit 10 is then programmed for a number of uses and the electronic key can be disposed.

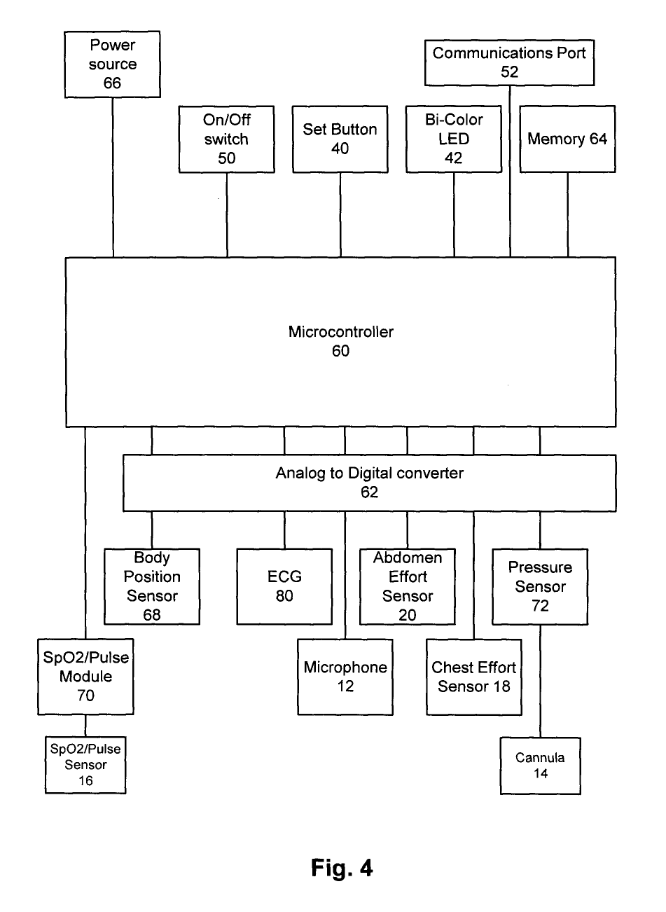

Referring again to FIG. 2, the processing and recording unit 10 contains a printed circuit board 54, which can be attached to a SpO2/pulse circuit module, as described below. Printed circuit board 54 includes a microprocessor, analog to digital (A/D) converters, flash memory, supporting computing circuitry, as described in greater detail below, and interfaces with the various connectors described above in relation to FIG. 1. FIG. 4 is a block diagram of circuitry of processing and recording unit 10. A/D converters 62 and microcontroller 60 reside on printed circuit board 54, where, in conjunction with memory 64, all of the audio sampling and sensor data measurement and storage is conducted. Compression algorithms, which are used to sample audio signals at frequencies of 1000 Hz or higher, are stored by memory 64 and utilized by A/D converters 62 and microcontroller 60 when necessary. Memory 64, which in a preferred embodiment is flash memory, is sufficient to store at least six hours of continuous sound data. Power source 66 powers A/D converters 62 and microcontroller 60, as well as the other components of the present invention. Communication port 52 can be used to download data to an external computing device from memory 64. A body position sensor 68, such as an accelerometer, can also be integrated into the device 10.

In order to demonstrate how the present invention operates to collect data on the various aspects of a sleep-related disorder, operation of the sleep disorder processing and recording unit of the present invention will now be described with reference to FIGS. 1-4.

A reduction or absence of airflow at the airway opening defines sleep-disordered breathing. One method of detecting such reduction or absence of airflow is to measure changes in pressure in the nasal airway that occur with breathing. This approach provides an excellent reflection of true nasal flow. A simple nasal cannula, such as nasal cannula 12, attached to a pressure transducer can be used to generate a signal. It also allows detection of the characteristic plateau of pressure due to inspiratory flow limitation that occurs in obstructive hypopneas.

A sleep disorder event, such as collapse of the upper airway, can be identified when, for example, the amplitude of the respiratory airflow and effort signals decrease by at least 50%, snoring sounds either crescendo or cease, and oxygen desaturation occurs. A respiratory event can, for example, be confirmed by the recognition of an arousal (i.e., the person awakens to breathe), typically identified by an increase in heart rate, or change in snoring pattern. Testing both before and after treatment allows a clinician to more accurately evaluate the results of their treatment on a subject. The best method for determining the success of sleep-related disorder treatments is through the measurement of a subject's breathing. Most clinicians rely on what is called the respiratory disorder index (number of respiratory events per hour), snoring index (number of snores per hour) and snoring magnitude. The use of auditory signals at high frequencies of 1000 Hz or more allows the clinician to determine the entire power spectrum of the auditory signal, and allow accurate characterization of the volume of the snoring in decibels. This yields a more accurate, quantitative result than current systems, which typically sample at 20 Hz-100 Hz, which cannot accurately provide a power spectrum characterizing the snoring due to the rapidly changing nature of a snoring signal.

Various sensors can collect different information related to each sleep disorder event. For example, an ECG sensor set can be used to determine the RR interval, commonly referred to as beats per minute, to assess cardiac function. Body position is normally classified as: right side, left side, supine, prone, or upright. A body position sensor can be used to determine if an airway collapse occurs only or mostly in just one position (typically supine). A microphone can be used to record sound amplitude and frequency, such as snoring and breath sounds.

Oxyhemoglobin, or blood oxygen, saturation (SpO2) can be determined using a pulse oximeter. A pulse oximeter uses two different light sources (e.g., red and infrared) to measure different absorption or reflection characteristics for oxyhemoglobin and deoxyhemoglobin. The oximeter then determines the ratio (percent) of saturated to unsaturated hemoglobin. Transmission oximetry devices are commonly used and operate by transmitting light through an appendage, such as a finger or an earlobe, and comparing the characteristics of the light transmitted into one side of the appendage with that detected on the opposite side. Another method to determine blood oxygen saturation is by reflectance oximetry, which uses reflected light to measure blood oxygen saturation. Reflectance oximetry is useful to measure oxygen saturation in areas of the patient's body that are not well suited for transmission measurement.

Respiratory effort can be determined by plethysmography. In plethysmography, the subject wears two elastic bands, one around the chest and the other around the abdominal area. Pressure transducers, such as piezo transducers, embedded in the bands can be used to detect chest expansion. Alternately, inductance plethysmography can be used to detect and monitor chest and abdominal respiratory effort. A conductive coil in each of these bands form part of an inductor in a tuned circuit. Sinusoidal signals are generated from an oscillator, and changes in cross-sectional area of the inductor result in a change in output frequency of the signal, hence the thoracic and abdominal cross-sectional area.

Audio (sound) data is generated by microphone 12 for sampling by A/D converters 62 and microcontroller 60. The sampling rate is preferably 1000 Hz or higher. SpO2/pulse sensor 16, cannula 14, chest effort sensor 18, abdomen effort sensor 20 and body position sensor 68 are all connected to A/D converters 62 and microcontroller 60 for the purpose of measuring the data collected by these devices. In the case of SpO2/pulse sensor 16 and cannula 14, there is an indirect connection through an SpO2/pulse module 70 and internal pressure sensor 72, respectively. The remaining components are all connected to A/D converters 62 and microcontroller 60 directly. The set button 40, two colour LED 42 and ON/OFF switch 50 are all preferably directly connected to the microcontroller.

Dual purpose auxiliary (AUX) connector 30 is used as the connector for audio microphone 12. Microphone 12 is capable of detecting breathing sounds of a person and as such is fastened adjacent a breathing airway of a subject. Microphone 12 generates signals which are then sent to an amplification and filtering circuit and then to the microprocessor on the printed circuit board 54 for sampling and storage. Printed circuit board 54 contains firmware that compresses any audio signal received so that the processing and recording unit 10 can preferably store at least six hours of audio data. There is also firmware and hardware that verifies integrity of the storing of data by time-stamping all information so that all data can be verified at any time as being accurate.

Leur lock connector 32 is used to connect a nasal or nasal/oral cannula 14 to the processing and recording unit 10. When a subject wearing nasal or nasal/oral cannula 14 inhales or exhales, the air pressure at the nose, or nose and mouth, is transmitted to a pressure conducting tube 44 which is connected to the internal pressure sensor module 72. The pressure measurements measured by the internal pressure sensor module 72 are used by the microprocessor to indicate airflow and to derive airflow output.

In the illustrated embodiments, the processing and recording unit 10 has two dual 1.5 mm safety pin keyed connections 36, 38 for measuring respiratory effort. Chest respiratory effort connector 36 is used to connect to a piezo effort sensor band 18 located on the chest. Abdomen respiratory effort connector 38 is used to connect to a piezo effort sensor band 20 located on the abdomen.

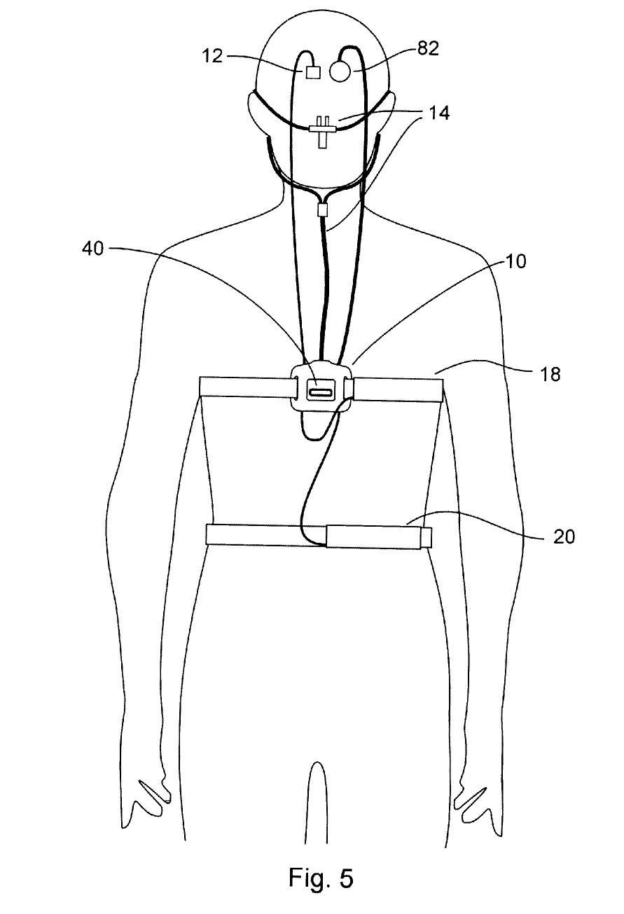

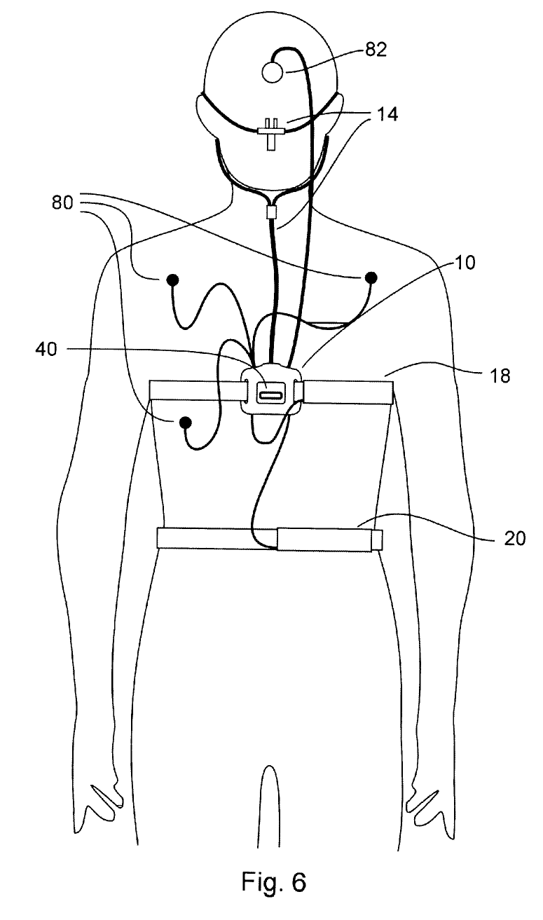

As shown in the embodiment of FIG. 5, connector 30 can also be used as an interface for a three lead ECG sensor 80 when the unit is used for cardiac measurement purposes. Although illustrated as a ECG sensor, one skilled in the art will appreciate that this element can be replaced with, or supplemented by, either or both of an EMG and an EEG. SpO2 connector 34 can be used to connect the transmission SpO2/pulse finger sensor 16 or a reflectance SpO2/pulse forehead sensor 82 to processing and recording unit 10. Through the use of SpO2/pulse circuit module 70, the processing and recording unit 10 can be used to collect oxyhemoglobin saturation levels and pulse in beats per minutes (bpm). When in this configuration, the processing and recording unit 10 preferably collects heart waveforms signals (ECG) at sampling rates of 100 Hz and higher. Three lead ECG sensor 80, cannula 14, chest effort sensor 18, body position sensor 68 and abdomen effort sensor 20 are again all connected to A/D converters 62 and microcontroller 60, directly or indirectly, for the purpose of measuring the data collected by these devices. Commercially available implementations of SpO2/pulse sensor 82 provide a digital output and thus do not require connection to A/D converter 62, although if an analog implementation of SpO2/pulse sensor 82 is employed, it can be connected to A/D converter 62 to provide microcontroller 60 with a digital signal. FIG. 6 shows yet another configuration of the diagnostic system of the present invention. A subject wearing the processing and recording unit 10 present invention configured with a forehead SpO2 sensor 82, and a microphone 12. As will be appreciated by those of skill in the art, any number of suitable sensors can be substituted for those shown in the illustrated embodiments, and multiple individualized configurations can be selected by a clinician in order to properly diagnose a given subject's sleep disorder condition. One skilled in the art will appreciate that although A/D converter 62 has been illustrated as a single element, multiple A/D converters can be used.

The monitoring and diagnostic device of the present invention can be provided as a standalone unit for use with preexisting sensors, or can be provided as a kit with various sensors. As a single use sensor kit, it is contemplated that the kit would include such items as a battery, cannula, hydrophobic filter, SpO2/pulse sensor, microphone, respiratory effort sensor bands, and customized foam tape for securing the SpO2 sensor to the subject's body

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A sleep disorder monitoring and diagnostic device, comprising:
a processing and recording unit to be worn by a subject for monitoring and diagnosis of a sleep disorder in the subject, the processing and recording unit having:
a plurality of connectors to permit reconfigurable attachment of various physiological sensors for sensing physiological conditions of the subject;
processing means for sampling and processing signals from the physiological sensors, at least one of the signals being sampled at a rate of at least about 1000 Hz; and
storage means for recording the sampled and processed signals.

2. The device of claim 1, wherein the plurality of connectors include two or more different connector types.

3. The device of claim 2, wherein the two or more different connector types are selected from the group consisting of leur lock connectors, auxiliary connectors, and pin keyed connectors.

4. The device of claim 1, wherein the physiological sensors include a microphone.

5. The device of claim 4, wherein a sound sample rate for the signal obtained from the microphone is at least 1000 Hz.

6. The device of claim 1, wherein the physiological sensors are selected from the group consisting of oxyhemoglobin sensors, pulse rate sensors, electrocardiogram (ECG) sensors, electroencephalography (EEG) sensors, electromyography (EMG) sensors and respiratory effort sensors.

7. The device of claim 1, further including an airflow pressure sensor for use with a nasal cannula.

8. The device of claim 1, further including a body position detector.

9. A sleep disorder and diagnostic device kit, comprising:
a plurality of physiological sensors for sensing physiological conditions of a subject; and
a processing and recording unit to be worn by a subject for monitoring and diagnosis of a sleep disorder in the subject, the processing and recording unit having a plurality of connectors to permit reconfigurable attachment of the plurality of physiological sensors, a processing means for sampling and processing signals from the physiological sensors, at least one of the signals being sampled at a rate of at least about 1000 Hz, and a storage means for recording the sampled and processed signals.

10. The kit of claim 8, wherein the kit is a single-use kit.

11. The kit of claim 8, wherein the plurality of connectors are selected from the group consisting of leur lock connectors, auxiliary connectors, and pin keyed connectors.

12. The kit of claim 8, wherein the plurality of physiological sensors include a microphone.

13. The device of claim 12, wherein a sound sample rate for the signal obtained from the microphone is at least 1000 Hz.

14. The kit of claim 8, wherein the plurality of physiological sensors are selected from the group consisting of oxyhemoglobin sensor, pulse rate sensors, electrocardiogram (ECG) sensors, electroencephalography (EEG) sensors, electromyography (EMG) sensors and respiratory effort sensors.

15. The kit of claim 8, wherein the processing and recording unit further includes an airflow pressure sensor for use with a nasal cannula.

16. The device of claim 1, wherein the processing and recording unit further includes a body position detector.

17. A sleep disorder monitoring and diagnostic method, comprising:
attaching a processing and recording unit to a subject, the processing and recording unit having a plurality of connectors to permit reconfigurable attachment of various physiological sensors;
connecting a plurality of physiological sensors to the processing and recording unit, including a microphone to detect sound related to breathing and snoring; and
sampling signals from the plurality of physiological sensors, including sampling sound, via the microphone, at at least 1000 Hz.
